(19) United States
(12) Patent Application Publication
(10) Pub. No.: US 2021/0145646 A1
(43) Pub. Date: May 20, 2021

(54) WOUND DRESSING WITH SATURATION INDICATOR

(71) Applicants: KCI USA, INC., San Antonio, TX (US); SYSTAGENIX WOUND MANAGEMENT, LIMITED, West Sussex (GB)

(72) Inventors: Sally STEPHENS, Skipton (GB); Leanna POOLE, Rawtenstall (GB); Alexander WAITE, Cowling (GB); Craig DELURY, Gargrave (GB)

(21) Appl. No.: 16/623,067

(22) PCT Filed: Jun. 13, 2018
(86) PCT No.: PCT/US2018/037363

§ 371 Date: Dec. 16, 2019

Related U.S. Application Data
(60) Provisional application No. 62/521,900, filed on Jun. 19, 2017.

(51) Int. Cl. A61F 13/00

(52) U.S. Cl. CPC A61F 13/00055; A61F 13/00987; A61F 13/00012; A61F 13/00068

(57) ABSTRACT

Described herein are wound dressings comprising: an absorbent layer capable of absorbing a given volume of fluid and comprising a first sorption zone comprising a first density of fibers and a second sorption zone comprising a second density of fibers higher than the first density; a hydrophilic foam layer coupled to the absorbent layer; and a backing layer extending beyond the absorbent layer and the hydrophilic foam layer to define a backing layer margin, wherein, upon exposure of the absorbent layer to a saturation threshold of wound exudate, the first sorption zone expands to a first thickness and the second sorption zone expands to a second thickness that is visually distinguishable from the first thickness. Methods of manufacturing the wound dressing and of eliminating, minimizing, or reducing edema are also described herein.

### CROSS-REFERENCE TO RELATED APPLICATIONS

This application claims the benefit of priority to U.S. Provisional Application No. 62/521,900, filed on Jun. 19, 2017, which is incorporated herein by reference in its entirety.

### TECHNICAL FIELD

The subject matter set forth in the appended claims relates generally to treating tissue, including wound dressings with a saturation indicator.

### BACKGROUND

A wide variety of materials and devices, generally characterized as “wound dressings,” are generally known in the art for use in treating an injury or other disruption of tissue. Such wounds may be the result of trauma, surgery, or disease, and may affect skin or other tissues. In general, wound dressings may control bleeding, absorb wound exudate, ease pain, assist in debriding the wound, protect wound tissue from infection, or otherwise promote healing and protect the wound from further damage.

Although the clinical benefits and advantages of wound dressings may be widely accepted, improvements to wound dressings may benefit healthcare providers and patients.

### BRIEF SUMMARY

New and useful compositions of dressing layers and wound dressings including such a dressing layer, methods for manufacturing same, and methods for using the same are set forth in the appended claims. Illustrative embodiments are also provided to enable a person skilled in the art to make and use the claimed subject matter.

Some examples of such compositions may comprise an absorbent layer capable of absorbing a given volume of fluid, a hydrophilic foam layer coupled to the absorbent layer, and a backing layer. The absorbent layer may comprise a first sorption zone having a first density of fibers and a second sorption zone having a second density of fibers higher than the first density. Upon exposure of the absorbent layer to a saturation threshold of liquid such as wound exudate, the difference in fiber density between the sorption zones can result in a difference in fluid expansion in the thickness direction. The thickness differences at the saturation threshold may be visually distinguishable, for example, to allow an external observer to visually observe the saturation threshold status of the absorbent layer. The backing layer may extend beyond the absorbent layer and the hydrophilic foam layer to define a margin.

Another example relates to a method for manufacturing a wound dressing composition. The wound dressing may, upon exposure to a saturation threshold of fluid, differentially expand in a visually distinguishable manner. The method may comprise providing a wound dressing that comprises a backing layer, a hydrophilic foam layer, and an absorbent layer capable of absorbing a given volume of fluid such as wound exudate. The method may also comprise differentially treating the absorbent layer using a needling apparatus to attain a first zone and a second zone having differential levels of needling treatment. Upon exposure of the absorbent layer to a saturation threshold of liquid such as wound exudate, the difference in needling between the first and second zones can result in a difference in fluid expansion in the thickness direction. The thickness differences at the saturation threshold may be visually distinguishable, for example, to allow an external observer to visually observe the saturation threshold status of the absorbent layer. The method may also comprise coupling the differentially treated absorbent layer to the foam layer, such that the backing layer extends beyond the absorbent layer and the hydrophilic foam layer to define a margin.

Another example relates to a method of eliminating, minimizing, or reducing post-operative edema for a wound surrounded by tissue. The method may comprise positioning a wound dressing described herein over the wound, such that at least a portion of the adherent layer of the wound dressing contacts the tissue.

Objectives, advantages, and one or more preferred modes of making and using the claimed subject matter may be understood by reference to the accompanying drawings, in conjunction with the following detailed description of illustrative embodiments.

### DRAWINGS

FIG. 1 is a perspective view in cross-section of a wound dressing according to this specification.

FIG. 2 is a diagram of the wound dressing of FIG. 1 with a therapy system.

FIG. 3A is a perspective view of an absorbent layer that may be associated with the wound dressing of FIG. 1. FIG. 3B is a section view of a wound dressing containing the absorbent layer of FIG. 3A. FIG. 3B also provides an expanded view of the absorbent layer of FIG. 3A with a given volume of fluid absorbed.

It should be noted that the figures set forth herein are intended to exemplify the general characteristics of materials and methods among those of the present invention, for the purpose of the description of certain embodiments. The figures may not precisely reflect all the characteristics of any given embodiment, and are not necessarily intended to define or limit specific embodiments within the scope of this invention.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

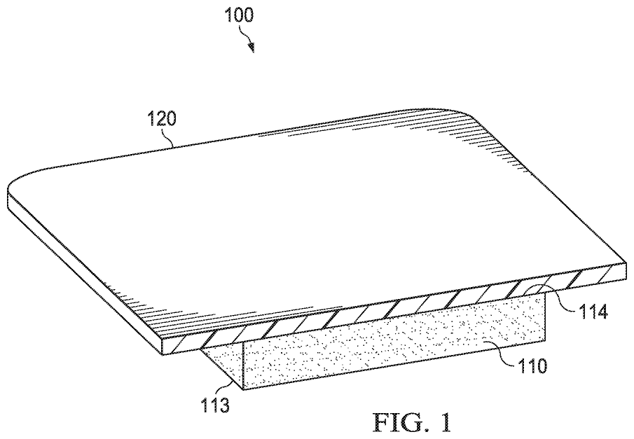

Disclosed herein are embodiments of a dressing layer, embodiments of wound dressings including such a dressing layer, and embodiments of therapy systems including same. Also disclosed herein are embodiments of methods related to (for example, methods of making, methods of using, etc.) the disclosed dressing layers, wound dressings, and therapy systems. For example, FIG. 1 illustrates an embodiment of a wound dressing 100. Generally, and as will be disclosed herein, the wound dressing 100 may be configured to provide therapy to a tissue site in accordance with the disclosure of this specification.

As used herein, “tissue site” is intended to broadly refer to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), skin flaps, and grafts, for example. The term “tissue site” may also refer to areas of any tissue that are not necessarily wounded or defective, but are instead areas in which it may be desirable to add or promote the growth of additional tissue, such as granulation. As noted above, the tissue site can refer to an area of tissue where prophylactic treatment is desired, even without a wound or defect, for example with respect to pressure ulcer prevention.

In some embodiments, a wound dressing may include one or more dressing layers configured to interface with the tissue site. For example, in the embodiment of FIG. 1, the wound dressing 100 may include a dressing layer 110. The dressing layer 110 may generally be configured to be positioned on, over, in, adjacent to, or otherwise in contact with (collectively, “near”) the tissue site.

### Dressing Layer

In various embodiments, the dressing layer 110 may be configured so as to be in contact with a portion of the tissue site, substantially all of the tissue site, or the tissue site in its entirety. If the tissue site is a wound, for example, the dressing layer 110 may partially or completely fill the wound, or may be placed over or near the wound. In various embodiments, the dressing layer 110 may take many forms, and may have many sizes, shapes, or thicknesses depending on a variety of factors, such as the type of treatment being implemented or the nature and size of a tissue site. For example, the size and shape of the dressing layer 110 may be adapted to the contours of deep and irregular shaped tissue sites, may be configured so as to be adaptable to a given shape or contour, or both. Moreover, in some embodiments, any or all of the surfaces of the dressing layer 110 may comprise projections or an uneven, course, or jagged profile that can, for example, induce strains and stresses on a tissue site, which may be effective to promote granulation at the tissue site.

In some embodiments, the dressing layer 110 may be in substantially sheet form. For example, the dressing layer 110 may comprise a generally planar structure having two opposite-facing planar surfaces and a depth or thickness orthogonal to the planar surfaces. More particularly, for example, the dressing layer 110 may comprise a first surface 113 and an opposite-facing second surface 114. The first surface 113 may be adapted to face a wound, having a surface area sufficient to cover an appropriate portion, if not all, of the wound. For example, a surface area from about 1 cm² to about 4000 cm² may be suitable for many applications. In various embodiments, the first surface 113 and the second surface 114 may have any suitable shape, examples of which include, but are not limited to, triangles, squares, rhombuses, rhomboids, diamonds, rectangles, trapezoids, ellipses, ellipsoids, circles, semi-circles, pie-wedges, ovals, and various polygons having four, five, six, seven, eight, or more sides. These shapes may additionally or alternatively be adaptations of such common shapes. In some embodiments, shapes with typically rounded edges may be altered to be flatter, such as a rounded hexagonal/octagonal shape made by flattening the rounded edges of a circle. Additionally or alternatively, shapes with typically rounded edges may be altered to be sharper, such as a tear-drop shape made by sharpening a rounded end of an ellipse or ellipsoid, or such as an eye shape made by sharpening two rounded, opposing ends of an ellipse or ellipsoid. Further additionally or alternatively, shapes with typically pointed edges may be altered to be more rounded, such as for a blunt-ended triangle. Still further additionally or alternatively, shapes with typically flat edges may be altered to be more rounded, such as by converting the flat sides of any regular polygon to a sinusoidal edge to form a doily shape with an undulating, curvy edge. The shape and area of the back surface 114 may be customized to the location and type of wound onto which the wound dressing 100 is to be applied.

### Dressing Layer Composition

There can be various embodiments of the composition of the dressing layer. In some embodiments, the dressing layer 110 may be a single layer, whereas, in some embodiments, the dressing layer 110 may represent a multi-layer composite structure. For example, the dressing layer 110 may comprise at least two layers coupled to each other.

In some embodiments, the dressing layer 110 may comprise an absorbent layer capable of absorbing a given volume of fluid and a hydrophilic foam layer coupled to the absorbent layer. The absorbent layer may comprise superabsorbent fibers, non-woven fibers, or a combination thereof. In various embodiments, the absorbent layer may comprise a first sorption zone comprising a first density of fibers and a second sorption zone comprising a second density of fibers higher than the first density. Upon exposure of the absorbent layer to a saturation threshold of liquid such as wound exudate, the difference in fiber density between the sorption zones can result in a difference in fluid expansion in the thickness direction. The thickness differences at the saturation threshold may be visually distinguishable, for example, to allow an external observer to visually observe the saturation threshold status of the absorbent layer.

In those embodiments, the difference between the first density and the second density may be based on a difference in degrees of needling treatment respectively applied to the first sorption zone and the second sorption zone, for example, by a needling apparatus such as a needling loom. Needling apparatuses can be used to knit to different types of fibrous materials such as non-wovens together, and they may additionally or alternatively be used to increase fiber density in a single fibrous material. In either case, one or more barbed needles can be punctured into/through the fibrous material(s). Differences in degrees of needling treatment can arise from different numbers or arrays of barbed needles, from different barbed needle puncture speeds or frequencies, from different sizes of barbs or needles, from the like, or from some combination thereof. Without being bound by theory, it is believed that the differential degree of needling treatment between the zones can result in a denser web of fibers in the second sorption zone. The increased fiber density may translate to a reduced tendency or capability, upon fluid absorption, for the absorbent fibers in the second sorption zone to expand as much as the absorbent fibers in the first sorption zone. This reduced tendency for expansion between the two zones can be noticed visually when the relative expansion, or lack thereof, is observed in the thickness direction. In this way, the differential thickness can visually indicate when a threshold level of fluid sorption has already taken place in the wound dressing. In some embodiments, the threshold level of fluid sorption, and thus a thickness differential, can advantageously correlate with a need to change the wound dressing. This correlation can be noticed by an external observer, such as a health care professional, a caretaker, or a patient, without the need to remove the dressing prematurely.

In various embodiments, the interface between the two sorption zones in the absorption layer can take any suitable or desired shape, message, or topological design. Non-limiting examples can include triangles, squares, rhombuses, rhomboids, diamonds, rectangles, trapezoids, ellipses, ellipsoids, circles, semi-circles, ellipses, ellipsoids, pie-wedges, ovals, cardioids, various polygons having four, five, six, seven, eight, or more sides, alphanumeric characters, symbols, company logos, fractal designs, reproductions of pictures, and the like, and combinations thereof. These shapes may additionally or alternatively be adaptations of such shapes. In some embodiments, shapes with typically rounded edges may be altered to be flatter, such as a rounded hexagonal/octagonal shape made by flattening the rounded edges of a circle. Additionally or alternatively, shapes with typically rounded edges may be altered to be sharper, such as a tear-drop shape made by sharpening a rounded end of an ellipse or ellipsoid, or such as an eye shape made by sharpening two rounded, opposing ends of an ellipse or ellipsoid. Further additionally or alternatively, shapes with typically pointed edges may be altered to be more rounded, such as for a blunt-ended triangle. Still further additionally or alternatively, shapes with typically flat edges may be altered to be more rounded, such as by converting the flat sides of any regular polygon to a sinusoidal edge to form a doily shape with an undulating, curvy edge.

Although the embodiments disclosed herein emphasize two different sorption zones with two different severities or extents of needling treatments, in some embodiments the second sorption zone may receive some needling treatment and the first sorption zone may receive no needling treatment. In most embodiments, the second sorption zone may simply be treated using a more severe needling regimen than applied the first sorption zone.

In some embodiments, the dressing layer 110 can be configured to exhibit substantially elastic recovery under wound treatment conditions. In various embodiments, however, at least one of the component layers of the wound dressing may not exhibit substantially elastic recovery under wound treatment conditions. For instance, in some embodiments, the dressing layer 110 may include a composite island structure, which may comprise an elastic foam layer coupled to an absorbent layer that is not elastic. In this way, the foam layer can enable the composite island, and thus the wound dressing, to exhibit substantially elastic recovery at wound treatment conditions, which may be a desirable characteristic in combination with an ability to absorb generous amounts of liquid.

In this context, a material that exhibits substantially elastic recovery under wound treatment conditions is termed “elastic”, and a material that does not exhibit substantially elastic recovery under wound treatment conditions is termed “not elastic”. For example, the dressing layer 110, having a width and a length perpendicular to the width, may be considered elastic if it exhibits at most about 1%, and advantageously about 0%, permanent deformation when subjected to about 20% strain, relative to the length, for about 15 minutes at that strain level, using an Instron™ mechanical testing machine, for example, at an initial imposed strain rate of about 1% elongation per minute up to the total strain value, at which point it can be held for the total strain time. Even though particular values are specified with respect to the strain test, materials, layers, or compositions may be considered elastic if optionally tested with one or more parametric deviations, including but not limited to: being conducted at greater than about 20% strain (for example, between about 20% strain and about 50% strain or between about 25% strain and about 40% strain), relative to the length; being conducted for greater than about 15 minutes (for example, between about 15 minutes and about 120 hours, between about 20 minutes and about 96 hours, or between about 30 minutes and about 48 hours) at the total strain level; and being conducted at an initial strain rate greater than 1% elongation per minute (for example, between about 1% elongation per minute and about 200% elongation per minute or between about 2% elongation per minute and about 100% elongation per minute).

Substantial elastic recovery may be particularly important for treating wounds in areas of relatively high articulation/flexure, such as proximal to shoulder, elbow, knee, ankle, and/or hip joints (particularly knee and/or elbow joints). It may be desirable for the wound dressing to be able to undergo significant local flexure and substantially retain its shape, and its contact with the wound site, upon significant articulation. Maintaining shape or wound contact can reduce the frequency of necessitated wound dressing changes, reduce tissue blistering from improper contact, increase wound exudate absorption, reduce wound healing time, increase patient comfort, or combinations thereof.

An absorbent layer is typically present in the dressing layer 110, either as the single layer or as one of multiple layers. For example, the dressing layer 110 may comprise a composite island having one or more absorbent layers. The absorbent layer may comprise a non-woven material of predominantly non-woven fibers, in some embodiments. For example, in various embodiments, the absorbent layer may comprise from about 50 parts to about 100 parts by weight of cellulosic (for example, cellulose ether) fibers and optionally up to 50 parts by weight of reinforcing fibers. In particular embodiments, the absorbent layer may comprise from about 50 parts to about 90 parts by weight, from about 60 parts to about 90 parts by weight, from about 65 parts to about 85 parts by weight, or from about 70 parts to about 90 parts by weight of cellulosic fibers and about 40 parts to about 10 parts by weight, from about 35 parts to about 15 parts by weight, from about 30 parts to about 10 parts by weight, from about 30 parts to about 15 parts by weight, or from about 25 parts to about 10 parts by weight of reinforcing fibers. In some optional embodiments, biodegradable or bioresorbable components may additionally be present in the absorbent layer, for example in amounts from about 1 part to about 20 parts by weight, such as from about 1 part to about 15 parts by weight or from about 1 part to about 10 parts by weight.

In some multi-layer embodiments, a bottom surface of the absorbent layer may be coupled to the top surface of the foam layer, whereas, in other multi-layer embodiments, a bottom surface of the foam layer may be coupled to the top surface of the absorbent layer.

When cellulosic fibers are present in the absorbent layer, they may be composed of at least one of carboxymethyl cellulose (CMC), carboxylethyl cellulose, hydroxyethyl cellulose, hydroxypropyl cellulose, hydroxypropylmethyl cellulose, and cellulose ethyl sulphonate (CES) (particularly carboxymethyl cellulose), for example. In some embodiments, the cellulosic component may be at least partially in a salt form, for example, comprising a physiologically acceptable cation such as sodium. CMC is commercially available from a variety of sources, such as under the tradenames Walocel™ (sold by The Dow Chemical Company) and Cekol® (sold by CP Kelco). When reinforcing fibers are present in the absorbent layer, for example, they may be composed of a polyurethane gel, an amide polymer such as Nylon 6,6, an olefin polymer such as HDPE, an ester polymer such as PET, a modified acrylamide polymer, or a combination or copolymer thereof. When biodegradable or bioresorbable components are present in the absorbent layer, for example, they may be composed of, but not limited to, an alginic acid, an alginate salt, chitosan, chitin, a guar gum, a locust bean gum, a xanthan gum, a karaya gum, gelatin, pectin, a starch derivative, a glycosaminoglycan, a galactomannan, a chondroitin salt, heparin, a heparin salt, collagen, oxidized regenerated cellulose (ORC), hyaluronic acid, a hyaluronate salt, or a combination thereof. For such listed salt components, they may include any reasonable counterions, such as sodium, calcium, ammonium, or the like, or combinations thereof. The biodegradable or bioresorbable component(s) can be, for example, in the form of a film or a foam, such as an open-cell foam, a reticulated foam, or combinations thereof. When in foam form, the average pore size may vary according to needs of a prescribed therapy, for example, from about 400 microns to about 600 microns). Other physico-chemical properties of biodegradable or bioresorbable components, such as tensile strength, may be chosen or manipulated, for example, to be suitable to needs of a prescribed wound therapy.

In some embodiments, particularly if biodegradable or bioresorbable components are included, the dressing layer 110 may be characterized as having some bioresorbable character or as exhibiting bioresorbability. “Bioresorbable,” “bioresorbability,” “biodegradable,” and “biodegradability” may individually or collectively refer to a characteristic of a material to disintegrate, degrade, or dissolve upon exposure to physiological fluids or processes, for example, when the dressing layer 110 is positioned with respect to a tissue site. For example, in some embodiments, the dressing layer 110 or a material from which the dressing layer 110 is formed may form a gel when contacted with an aqueous medium, such as water, saline, blood, or wound exudate. Such bioresorbability may be exhibited as a result of chemical process or condition, a physical process or condition, or some combination thereof. For example, the bioresorbable characteristics of the dressing layer 110 may substantially reduce or eliminate the need to remove the dressing layer 110 from a tissue site to which it is applied. In some embodiments, at least about 90% by weight of the biodegradable or bioresorbable component (particularly at least about 95% by weight, at least about 99% by weight, or about 100% by weight) may be disintegrated, degraded, or dissolved with in a time period of from about 15 days to about 24 hours (particularly from about 12 days to about 36 hours or from about 10 days to about 48 hours), from introduction into a physiological environment when incubated with simulated physiological fluid at a temperature of about 37° C.

### Optional Wound Layer Additives

In some embodiments, the wound dressing, and particularly the wound layer, may optionally comprise one or more additional materials. Such optional components may include, for example, active materials such as preservatives, stabilizing agents, plasticizers, matrix strengthening materials, dyestuffs, and combinations thereof.

Additionally or alternatively, the wound dressing, and particularly the wound layer, may comprise one or more additional active materials, for example, antimicrobial agents which may be effective to aid in wound healing. Non-limiting examples of such active materials may include non-steroidal anti-inflammatory drugs such as acetaminophen, steroids, antimicrobial agents such as penicillins or streptomycins, antiseptics such as chlorhexidine, growth factors such as fibroblast growth factor or platelet derived growth factor, and other well-known therapeutic agents, alone or in combination. When present, such active materials may typically be included at any effective level that show therapeutic efficacy, while preferably not being at such a high level as to significantly counteract any critical or desired physical, chemical, or biological property of the wound dressing. Depending upon the therapeutic goal(s), the active material(s) may be loaded at a level of from about 10 wppm to about 10 wt % of the layer(s) in which it(they) are present, for example, from about 50 wppm to about 5 wt % or from about 100 wppm to about 1 wt %.

In some embodiments, the antimicrobial agents may comprise a safe and effective amount of poly(hexamethylene biguanide) (“PHMB”), which is also known as polyaminopropyl biguanid (“PAPB”) and polyhexanide, having the following general formula.

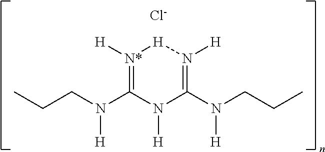

PHMB can be a cationic broad spectrum antimicrobial agent. PHMB may be synthesized by a variety of methods, including polycondensation of sodium dicyanamide and hexamethylenediamine. PHMB is commercially available from a variety of sources. In some embodiments, the PHMB may be present in one or more of the wound layers at a level of from about 0.005 wt % to about 0.025 wt % of each layer in which it is present, particularly from about 0.007 wt % to about 0.2 wt % or from about 0.008 wt % to about 0.012 wt %, or in some cases at about 0.01 wt %. In alternative embodiments, silver compounds having antimicrobial efficacy may completely or partially replace the PHMB, as desired.

In some embodiments where CMC is not already present, the composition may comprise CMC as a modifier for one or more characteristics of the wound dressing or dressing layer(s), for example, the rheological, absorbency, and other structural characteristics. CMC may be present in the layer(s) at any level appropriate to result in the desired absorbency, rheological, or other structural characteristics of the wound dressing.

In some embodiments, the dressing layer may contain a strengthening material, which can improve the handling characteristics of the wound dressing, for example, by decreasing its susceptibility to tearing. In a particular embodiment, the strengthening material may comprise non-gelling cellulose fibers. Such “non-gelling” cellulose fibers may be substantially water insoluble and may be produced from cellulose that has not been chemically modified to increase water solubility, for example, as contrasted from carboxymethyl cellulose or other cellulose ethers. Non-gelling cellulose fibers are commercially available, such as under the tradename Tencel® (sold by Lenzing AG). In some embodiments, such fibers may be processed from a commercially-available continuous length, by cutting into lengths from about 0.5 to about 5 cm or from about 2 to about 3 cm in length. The non-gelling cellulose fibers may be present in the composition at any level appropriate to result in the desired physical characteristics of the composition. In general, when present, the non-gelling cellulose fibers may comprise from about 1% to about 55% of the layer by weight, particularly from about 5% to about 40% of the layer by weight or from about 10% to about 25% of the layer by weight. In some embodiments, when present, the non-gelling cellulose fibers can be characterized as an additional or alternative reinforcing fiber and can be present in reinforcing fiber amounts.

### Wound Dressing—Cover (Backing Layer)

In most embodiments, aside from dressing layer 110, the wound dressing 100 may comprise one or more additional layers. In various embodiments, such additional layers may perform any of a variety of functions including, for example, adherence of the wound dressing to a tissue site or to surrounding tissues, increasing structural rigidity of the wound dressing, protection from moisture or other contaminants in the external environment, protection of a wound surface, delivery of one or more active or other materials to the wound surface, or combinations thereof. In various embodiments, the additional layers may be conformable to a wound surface or to the surrounding tissues, for example, being capable of conforming such that the appropriate surfaces of the wound dressing are in substantial contact with the wound or the surrounding tissues.

For example, in the embodiment of FIG. 1, the wound dressing 100 comprises a backing layer 120, which may be positioned over the dressing layer 110, for example, so as to cover the dressing layer 110 at the tissue site. The backing layer 120 may have a top surface and a bottom surface. The backing layer 120 may support the dressing layer 110 on the bottom surface of the backing layer 120, for example, such that a top surface of the dressing layer 110 can be proximate to the bottom surface of the backing layer 120. In some embodiments, the top surface of the dressing layer may be in contact with and adhered to the bottom surface of the backing layer 120.

In particular embodiments, the backing layer 120 of the wound dressing 100 may extend beyond the boundaries or edges of the dressing layer 110, so as to exhibit an exposed backing layer margin, which may typically be exhibited on the bottom side of the backing layer 120.

In some embodiments, the backing layer 120 may generally be configured to provide a barrier to microbes, a barrier to external contamination, and protection from physical trauma. For example, the backing layer 120 may be constructed from a material that can reduce evaporative losses and provide a fluid seal between two components or two environments, such as between a therapeutic environment and a local external environment. The backing layer 120 may be formed from a suitable material, such as a polymer, for example, which may comprise or be an elastomeric film or membrane that can provide a seal at a tissue site for a given negative-pressure source. In some embodiments, the backing layer 120 may comprise or be a polyurethane. In some embodiments, the backing layer 120 may have a high moisture-vapor transmission rate (MVTR). For example, in such an embodiment, the MVTR may be at least 300 g/m² per twenty-four hours. For example, the backing layer 120 may comprise a polymer drape, such as a polyurethane film, that may be permeable to water vapor but generally impermeable to liquid water. In some embodiments, the backing or drape may have a thickness in the range of about from 15 to about 50 microns.

In various embodiments, the backing layer 120 may have appropriate properties of expandability, resiliency, etc., such that the thickness differential exhibited by the different sorption zones of the absorbent layer be and remain observable and visually distinguishable. In such embodiments, the capability for an observer to visually distinguish differential thickness expansion should neither be unduly obscured by the backing layer nor by any other layers disposed between the absorbent layer and the backing layer.

### Wound Dressing—Secondary Layer

Additionally, in some embodiments, the wound dressing 100 may further comprise a secondary layer, for example, positioned between the dressing layer 110 and the backing layer 120. In some embodiments, the secondary layer may comprise fluid pathways interconnected so as to improve distribution or collection of fluids. For example, in some embodiments, the secondary layer may be a porous foam material having a plurality of interconnected cells or pores, for example, cellular foam, open-cell foam, reticulated foam, porous tissue collections, and other porous material such as gauze or felted mat generally include pores, edges, or walls adapted to form interconnected fluid pathways (for example, channels). For example, in some embodiments, the secondary layer may be a foam having pore sizes in a range of 400-600 microns. In one non-limiting example, the secondary layer may be an open-cell, reticulated polyurethane foam.

In some embodiments where the secondary layer is present, the secondary layer may be characterized as exhibiting absorbency. For example, the secondary layer may exhibit an absorbency of at least 3 g saline/g, particularly at least 5 g saline/g, from 5 to 50 g saline/g, from 8 to 40 g saline/g, or from 8 to 20 g saline/g. In some embodiments, the secondary layer may be hydrophilic. In an example in which the secondary layer may be hydrophilic, the secondary layer may also absorb (for example, wick) fluid away from a dressing layer 110. In such an embodiment, the wicking properties of the secondary layer may draw fluid away from dressing layer 110 by capillary flow or other wicking mechanisms. An example of a hydrophilic foam is a polyvinyl alcohol, open-cell foam. Other hydrophilic foams may include those made from or containing a polyether or a polyurethane. Other foams that may exhibit hydrophilic characteristics include hydrophobic foams that have been treated or coated to provide hydrophilicity.

### Other Optional Dressing Layers

In some embodiments, the top surface of the dressing layer may be in contact with and adhered to the bottom surface of the backing layer 120. This adherence may, in some embodiments, result from an adherent layer disposed between the dressing layer 110 and the backing layer 120, thus constituting direct adherence. Such direct adherence means that the adherent layer, or at least the portion disposed between the dressing layer 110 and the backing layer 120, can be comprised of one or more different kinds of physical or chemical adhesive compositions. Though wound-facing, the adherent layer or portion thereof, in some embodiments, would not be expected to directly contact the wound site and/or tissue proximal thereto. Nevertheless, in embodiments with a backing layer margin extending beyond the dressing layer 110, the adherent layer may typically extend out to cover all or part of the backing layer margin. In such embodiments, the portion of the adherent layer on the margin would likely adhere the wound dressing 100 to a wound site or tissue proximal thereto. Adherents that may directly contact tissue or that may be exposed to a wound environment can typically have additional requirements, such as biocompatibility, and therefore may be selected from a smaller list of physical or chemical adhesive compositions.

Adherence between the dressing layer 110 and the backing layer 120 may additionally or alternatively be indirect. For example, in some embodiments with a backing layer margin extending beyond the dressing layer 110, the adherent layer may be disposed on the backing layer margin and extend further over some portion of the dressing layer 110, such as the margin of the dressing layer but typically not its entirety. When this occurs without an adherent layer between the backing layer 120 and the dressing layer 110, the adherent layer may be said to indirectly adhere those layers, because those layers are each adhered to the adherent layer but not directly to each other. Such a configuration can allow an absorbent portion of the dressing layer 110 to expand differentially from the backing layer 120, for instance enabling relatively high levels of absorption of fluid with additional degrees of freedom. When the backing layer 120 is directly adhered to the dressing layer 110, the only ways for the absorbent portion of the dressing layer 110 to expand would be into the wound site and against the tissue, which can in some embodiments create undesirable wound site pressure.

In particular embodiments, the adherent layer can comprise a hydrocolloid material.

In some embodiments, absorbent material may be absent in or removed from a zone within the absorption layer. Such embodiments offer an additional or alternative mechanism enabling at least partial fluid absorptive expansion within the absorption layer, which can enable additional degrees of freedom for fluid absorption while creating no or little additional pressure on the wound site. For example, by creating a central zone in the absorbent layer of dressing layer 110 that is absent of material, the other portions of the absorbent layer can have extra volume to expand and can optionally experience increased fluid flow within the dressing layer 110, thus rendering the absorbent layer more efficient.

In some embodiments, the absorbent layer may be perforated to increase fluid flow, to reduce time to equilibrium absorption, or both. Such embodiments offer another additional or alternative mechanism enabling additional degrees of freedom for fluid absorption while creating no or little additional pressure on the wound site.

In some embodiments, the wound dressing 100 may include a non-adherent layer, disposed over the bottom portion of the dressing layer 110, opposite the backing layer 120. Non-adherent wound contacting layers may be particularly advantageous in fibrinous wound situations to reduce potential adherence of the dressing layer to the wound site, to enable fluid to be effectively drawn away from the wound site through the non-adherent layer, or both. In some embodiments, therefore, the non-adherent wound contacting layer may be perforated, for example, for increased fluid flow. In various embodiments, the non-adherent layer may comprise at least one of: an alkyl acrylate polymer, such as a methyl acrylate polymer, an ethyl acrylate polymer, or the like; an alkacrylate polymer, such as a methacrylate polymer, an ethacrylate polymer, or the like; and an alkyl alkacrylate polymer, such as a methyl methacrylate polymer, an ethyl methacrylate polymer, a methyl ethacrylate polymer, an ethyl ethacrylate polymer, or the like. Such (alk)acrylate polymers may be homopolymers but are more often copolymers, for example, with olefin comonomers. In particular, the non-adherent layer may comprise an ethylene-methyl acrylate copolymer, such as used in TIELLE™ and TIELLE™ Lite Dressings and in SILVERCEL™ NON-ADHERENT Dressings available from Systagenix Wound Management, Limited.

### Negative-Pressure Therapy

Additionally, in some embodiments, a dressing layer such as dressing layer 110, or a wound dressing comprising such a dressing layer, such as wound dressing 100, may be employed in therapy, for example to treat a tissue or wound site with reduced pressure. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, micro-deformation of tissue at a wound site, and combinations thereof. Individually or together, these benefits may increase development of granulation tissue and reduce healing times.

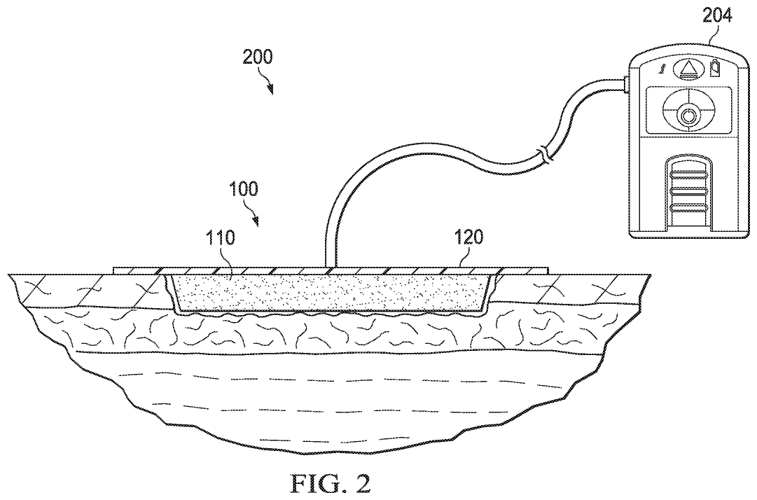

For example, FIG. 2 illustrates an embodiment of a negative-pressure therapy system 200 in a simplified schematic. Generally, the negative-pressure therapy system 200 may be configured to provide negative-pressure to a tissue site. In various embodiments, a negative-pressure therapy system may generally include a negative-pressure supply, and may include or be configured to be coupled to a distribution component. In general, a distribution component may refer to any complementary or ancillary component configured to be fluidly coupled to a negative-pressure supply in a fluid path between a negative-pressure supply and a tissue site. For example, in the embodiment of FIG. 2, the wound dressing 100 is fluidly coupled to a negative-pressure source 204, such that negative pressure may be applied to a tissue site via the wound dressing 100.

For example, the dressing layer 110 may be generally configured to distribute negative pressure, for example, so as to collect fluid. For example, in some embodiments, the dressing layer 110 may comprise or be configured as a manifold. A “manifold” in this context generally includes any composition or structure providing a plurality of pathways configured to collect or distribute fluid across a tissue site under pressure. For example, a manifold may be configured to receive negative pressure from a negative-pressure source and to distribute negative pressure through multiple apertures or pores, which may have the effect of collecting fluid and drawing the fluid toward the negative-pressure source. More particularly, in the embodiment of FIG. 2, the dressing layer 110 is configured to receive negative pressure from the negative-pressure source 204 and to distribute the negative pressure through the dressing layer 110. For example, this may have the effect of collecting fluid from a sealed space, such as by drawing fluid from the tissue site through the dressing layer 110. In additional or alternative embodiments, the fluid path(s) may be reversed or a secondary fluid path may be provided to facilitate movement of fluid across a tissue site. In some embodiments, the fluid pathways of a manifold may be interconnected to improve distribution or collection of fluids. In some embodiments, a manifold may comprise or be a porous foam material having a plurality of interconnected cells or pores. For example, cellular foams, open-cell foams, and reticulated foams generally include pores, edges, walls, or combinations thereof that may form interconnected fluid pathways, such as channels.

The fluid mechanics associated with using a negative-pressure source to reduce pressure in another component or location, such as within a sealed therapeutic environment, may be mathematically complex. However, the basic principles of fluid mechanics applicable to negative-pressure therapy are generally well-known to those skilled in the art. Herein, the process of reducing pressure may be described generally and illustratively herein as “delivering,” “distributing,” or “generating” negative pressure, for example.

In general, a fluid such as wound fluid flows toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. This orientation is generally presumed for purposes of describing various features and components herein. However, the fluid path may also be reversed in some applications, such as by substituting a positive-pressure source for a negative-pressure source, and this descriptive convention should not be construed as a limiting convention.

As used herein, “negative pressure” is generally intended to refer to a pressure less than a local ambient pressure, such as the ambient pressure in a local environment external to a sealed therapeutic environment provided by the wound dressing 100. In many cases, the local ambient pressure may also be the atmospheric pressure proximate to or about a tissue site. Additionally or alternatively, the pressure may be less than a hydrostatic pressure associated with the tissue at the tissue site. Unless otherwise indicated, values of pressure stated herein are gauge pressures. Similarly, references to increases in negative pressure typically refer to a decrease in absolute pressure, for example a more negative pressure, while decreases in negative pressure typically refer to an increase in absolute pressure, for example a less negative pressure or a more positive pressure. While the amount and nature of negative pressure applied to a tissue site may vary according to therapeutic requirements, the pressure is generally a low vacuum, also commonly referred to as a rough vacuum, for example between −5 mm Hg (−667 Pa) and −500 mm Hg (−66.7 kPa). Common therapeutic ranges can be between −75 mm Hg (−9.9 kPa) and −300 mm Hg (−39.9 kPa).

In various embodiments, a negative-pressure supply, such as the negative-pressure source 204, may be a reservoir of air at a negative pressure. Alternatively, negative-pressure source 204 may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components that further facilitate therapy, such as sensors, processing units, alarm indicators, memory, databases, software, display devices, or user interfaces. For example, in some embodiments, the negative-pressure source 204 may be combined with a controller and other components into a therapy unit. A negative-pressure supply may have one or more supply ports configured to facilitate coupling and de-coupling of the negative-pressure supply to one or more distribution components.

In various embodiments, components may be fluidly coupled to each other to provide a path for transferring fluids, as liquid or gas, between the components. For example, components may be fluidly coupled through a fluid conductor, such as a tube. As used herein, the term “fluid conductor” is intended to broadly include a tube, pipe, hose, conduit, or other structure with one or more lumina adapted to convey a fluid between two ends thereof. Typically, a fluid conductor may be an elongated, cylindrical structure with some flexibility, but the geometry and rigidity may vary. In some embodiments, the negative-pressure source 204 may be operatively coupled to the wound dressing 100 via a dressing interface. For example, in the embodiment of FIG. 2, the wound dressing 100 may be coupled to the negative-pressure source 204 via a dressing interface to receive negative pressure.

### Methods of Manufacture

Also disclosed herein are methods for manufacturing a wound dressing which differentially expands in a visually distinguishable manner upon exposure to a saturation threshold of fluid. In most embodiments, the method may comprise providing a wound dressing, which may comprise a backing layer, a hydrophilic foam layer, and an absorbent layer capable of absorbing a given volume of liquid, such as wound exudate. The method can then include differentially treating various areas of the absorbent layer using a needling apparatus, such as a needling loom, to form at least a first needled zone and a second needled zone. Needling apparatuses may be used to knit to different types of fibrous materials such as non-wovens together, and they may additionally or alternatively be used to increase fiber density in a single fibrous material. In either case, one or more barbed needles can be punctured into/through the fibrous material(s). Differences in degrees of needling treatment can arise from different numbers or arrays of barbed needles, from different barbed needle puncture speeds or frequencies, from different sizes of barbs or needles, from the like, or from some combination thereof. Without being bound by theory, it is believed that the differential degree of needling treatment between the zones can result in a denser web of fibers in the second sorption zone. The increased fiber density may translate to a reduced tendency or capability, upon fluid absorption, for the absorbent fibers in the second sorption zone to expand as much as the absorbent fibers in the first sorption zone. This reduced tendency for expansion between the two zones can be noticed visually when the relative expansion, or lack thereof, is observed in the thickness direction. In this way, the differential thickness can visually indicate when a threshold level of fluid sorption has already taken place in the wound dressing. In some embodiments, the threshold level of fluid sorption, and thus a thickness differential, can advantageously correlate with a need to change the wound dressing. This correlation can be noticed by an external observer, such as a health care professional, a caretaker, or a patient, without the need to remove the dressing prematurely. The method can then further comprise coupling the differentially treated absorbent layer to the foam layer to form a collective dressing layer, and also orienting the backing layer to extend beyond the collective wound dressing to define a wound backing layer margin. Optionally but typically, an adherent layer can be disposed on at least the backing layer margin.

### Methods of Use

Also disclosed herein are methods of treating a wound, for example, in the context of various therapies, such as eliminating, minimizing, or reducing post-operative edema, particularly in areas of relatively high articulation or flexure. Non-limiting examples of areas implicating relatively high articulation or flexure include shoulder, elbow, knee, ankle, or hip joints, particularly knee or elbow joints.

In some embodiments, a therapy method may comprise positioning the dressing layer 110 with respect to the tissue site. For example, in operation, the dressing layer 110 may be positioned proximate to the wound. The dressing layer 110 may be used with any of a variety of wounds, such as those occurring from trauma, surgery, or disease. For example, such wounds may be chronic wounds, venous ulcers, decubitus ulcers, or diabetic ulcers. For example, the dressing layer 110 may be placed within, over, on, or otherwise proximate to the tissue site. Additionally, in some embodiments, a cover sheet, for example, backing layer 120, may be placed over the dressing layer 110 and the backing layer 120 sealed to an attachment surface near the tissue site. For example, the backing layer 120 may be sealed to undamaged epidermis peripheral to a tissue site. In some embodiments, the dressing layer 110 may be positioned first and, after the dressing layer 110 has been positioned, the backing layer 120 may be positioned. In some other embodiments, the dressing layer 110 and backing layer 120 may be preassembled, for example, such that the dressing layer 110 and backing layer 120 are positioned with respect to each other prior to placement proximate the tissue site. Thus, the backing layer 120 can provide a sealed therapeutic environment including the dressing layer 110 and proximate to a tissue site, substantially isolated from the external environment.

Additionally, in some negative-pressure therapy embodiments, a negative-pressure therapy may comprise positioning the dressing layer 110 and backing layer 120 proximate to the tissue site, for example, a wound. For example, the various components of the dressing layer 110 may be positioned with respect to the tissue site sequentially or, alternatively, may be positioned with respect to each other and then positioned with respect to the tissue site. The negative-pressure therapy may further comprise sealing the dressing layer 110 to tissue surrounding the tissue site to form a sealed space. For example, the backing layer 120 may be placed over the dressing layer 110 and sealed to an attachment surface near the tissue site, for example, to undamaged epidermis peripheral to a tissue site. Thus, the dressing layer 110 and backing layer 120 can provide a sealed therapeutic environment proximate to the tissue site, substantially isolated from the external environment.

The negative-pressure therapy method may further comprise fluidly coupling a negative-pressure source to the sealed space and operating the negative-pressure source to generate a negative pressure in the sealed space. For example, the negative-pressure source 204 may be coupled to the wound dressing 100 such that the negative-pressure source 204 may be used to reduce the pressure in the sealed space. For example, negative pressure applied across the tissue site, for example via the wound dressing 100, may be effective to induce macrostrain and microstrain at the tissue site, as well as to remove exudates and other fluids from the tissue site.

### EMBODIMENTS

Additional or alternative examples may include one or more of the following embodiments.

Embodiment 1. A wound dressing comprising: an absorbent layer capable of absorbing a given volume of fluid and comprising a first sorption zone comprising a first density of fibers and a second sorption zone comprising a second density of fibers higher than the first density; a hydrophilic foam layer coupled to the absorbent layer; and a backing layer extending beyond the absorbent layer and the hydrophilic foam layer to define a backing layer margin, wherein, upon exposure of the absorbent layer to a saturation threshold of wound exudate, the first sorption zone expands to a first thickness and the second sorption zone expands to a second thickness that is visually distinguishable from the first thickness.

Embodiment 2. The wound dressing of embodiment 1, wherein an interface between the first sorption zone and the second sorption zone creates a topological design based on a differential between the first thickness and the second thickness.

Embodiment 3. The wound dressing of embodiment 1 or embodiment 2, wherein: the hydrophilic foam layer is substantially elastically deformable under wound treatment conditions, the absorbent layer is not elastically deformable under wound treatment conditions, and the coupled combination of the hydrophilic foam layer and the absorbent layer is substantially elastically deformable under wound treatment conditions.

Embodiment 4. The wound dressing of any of embodiments 1-3, wherein the absorbent layer comprises: (i) from about 45% to about 90% of cellulose ether fibers; and (ii) from about 10% to about 55% of reinforcing fibers.

Embodiment 5. The wound dressing of embodiment 4, wherein the cellulose ether fibers are composed of at least one of carboxymethyl cellulose, carboxylethyl cellulose, hydroxyethyl cellulose, hydroxypropyl cellulose, and hydroxypropylmethyl cellulose.

Embodiment 6. The wound dressing of embodiment 4 or embodiment 5, wherein the reinforcing fibers are composed of a polyurethane gel, an amide polymer, an olefin polymer, an ester polymer, a modified acrylamide polymer, or a combination or copolymer thereof.

Embodiment 7. The wound dressing of any of embodiments 4-6, wherein the absorbent layer further comprises from about 10% to about 40% of an alginate, chitosan, chitin, a guar gum, pectin, a starch derivative, a cellulose derivative, a glycosaminoglycan, a galactomannan, a chondroitin salt, heparin, a heparin salt, collagen, oxidized regenerated cellulose (ORC), hyaluronic acid, a hyaluronic acid salt, or a combination thereof.

Embodiment 8. The wound dressing of any of embodiments 1-7, wherein the foam layer is coupled to the absorbent layer by lamination.

Embodiment 9. The wound dressing of any of embodiments 1-8, wherein the backing layer is non-adherent and comprises a polyurethane.

Embodiment 10. The wound dressing of any of claims 1-9, wherein the absorbent layer comprises superabsorbent polymer fibers and/or non-woven fibers.

Embodiment 11. The wound dressing of any of embodiments 1-10, further comprising a non-adherent layer disposed on the foam layer opposite the absorbent layer.

Embodiment 12. The wound dressing of embodiment 11, wherein the non-adherent layer is perforated and comprises at least one of an alkyl acrylate polymer, an alkacrylate polymer, and an alkyl alkacrylate polymer.

Embodiment 13. The wound dressing of embodiment 12, wherein the alkyl acrylate polymer comprises an ethylene-methyl acrylate copolymer.

Embodiment 14. The wound dressing of any of claims 1-13, comprising an adherent layer coupled to the backing layer on at least a margin thereof, and optionally extending over a margin of the foam layer thereby fastening the adherent layer to the margin of the foam layer.

Embodiment 15. The wound dressing of any of embodiments 1-14, wherein the adherent layer comprises a hydrocolloid.

Embodiment 16. The wound dressing of any of claims 1-15, wherein a difference between the first density and the second density is based on a difference in degrees of needling respectively applied to the first sorption zone and the second sorption zone.

Embodiment 17. A method for manufacturing a wound dressing which, upon exposure to a saturation threshold of fluid, differentially expands in a visually distinguishable manner, the method comprising: providing a backing layer, a hydrophilic foam layer, and an absorbent layer capable of absorbing a given volume of wound exudate; differentially treating the absorbent layer using a needling apparatus, so as to attain a first needled zone and a second needled zone, each needled zone having differential needling levels such that, upon exposure to a saturation threshold of wound exudate, the first needled zone expands to a first thickness and the second needled zone expands to a second thickness that is visually distinguishable from the first thickness; and coupling the differentially treated absorbent layer to the foam layer, such that the backing layer extends beyond the absorbent layer to define a margin.

Embodiment 18. A method of reducing edema for a wound surrounded by tissue, the method comprising positioning the wound dressing according to any of claims 14-16, or a wound dressing made according to the method of claim 17, over the wound, optionally such that at least a portion of the adherent layer contacts the tissue.

Embodiment 19. The method of embodiment 18, wherein the wound is proximal to a knee joint or an elbow joint.

Embodiment 20. The method of embodiment 18 or embodiment 19, further comprising: sealing the wound dressing within a sealed space adjacent to the wound; and applying a negative pressure to the sealed space.

### EXAMPLES

One, some, or all of the advantages associated with the disclosed compositions, wound dressings, methods of making, and methods of using or treating may be further demonstrated by the following, non-limiting examples.

### Example 1

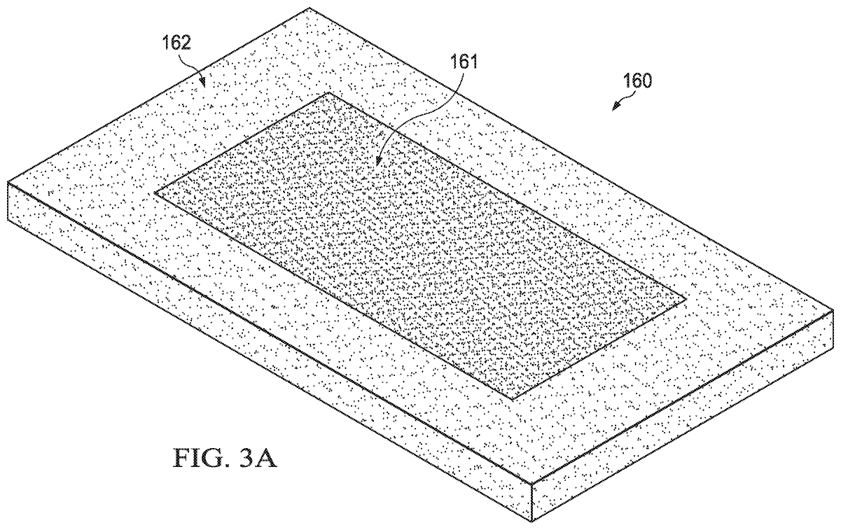

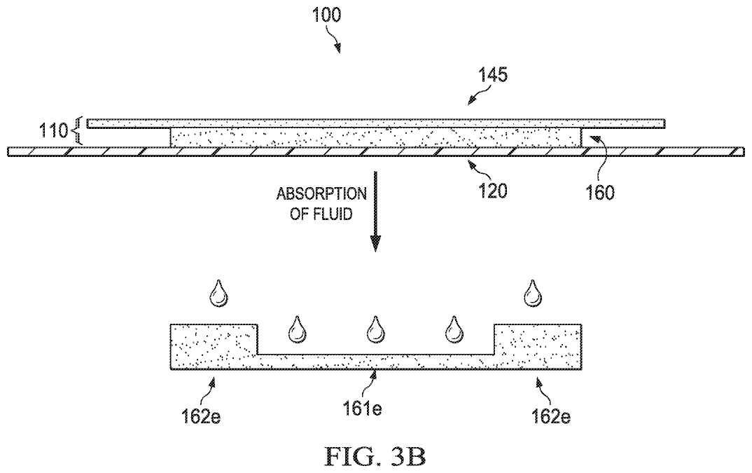

FIG. 3A shows an exemplary absorbent layer 160 having first 162 and second 161 sorption zones. In FIG. 3A, the second sorption zone 161 is represented by the central rectangle, while the first sorption zone 162 is represented by the margin framing that central rectangle. These two zones have been created by differential treatment by a needling apparatus to increase the fiber entanglements, or fiber density, proportionally more in the second sorption zone 161 than in the first sorption zone 162. FIG. 3B shows a section view of an exemplary wound dressing 100 with a composite dressing layer 110. FIG. 3B also identifies a polyurethane backing layer 120 of the wound dressing. As can be seen in FIG. 3B, dressing layer 110 is a multi-layer composite comprised of a CMC-based non-woven absorbent layer 160, having first 162 and second 161 sorption zones, coupled (in this case, laminated) to an open cell polyurethane foam layer 145. FIG. 3B also shows an expanded view of the absorbent layer 160 upon absorption of an amount of fluid near or at its threshold, for instance, its saturation point. In this expanded view of FIG. 3B, the thickness difference between the expanded first sorption zone 162e and the expanded second sorption zone 161e can be distinguishably observed, visually. Though not shown in FIG. 3B, the thickness difference can be visually distinguished even through the backing layer 120 of the wound dressing 100.

Optionally, the wound dressing 100 may further include a non-adherent layer (not shown, optionally perforated), disposed on the opposite side of foam layer 145 from absorbent layer 160. The non-adherent layer can face a wound site and can function to enable fluid, such as wound exudate, to be effectively drawn away from the wound site through the non-adherent layer. Though this Example shows the foam layer 145 closer to the wound site surface than the absorbent layer 160, the opposite configuration is also possible and envisioned. Also not shown but typically present is an adherent layer on at least a margin of the backing layer 120 to allow removable adherence to tissue, such as proximal to a wound site.

Though the second sorption zone 161 has a simple rectangular border in FIGS. 3A-3B, it is envisioned that the boundaries between the second sorption zone 161 and the first sorption zone 162 can represent any reasonable shape, design, message, picture, or logo.

### Non-Limiting Discussion of Terminology

The description and specific examples above are provided for illustration only and are not intended to limit the scope of the claimed subject matter. Moreover, recitation of multiple embodiments having stated features does not exclude other embodiments having additional features, or other embodiments incorporating different combinations of the stated features. Components may also be combined or eliminated in various configurations for purposes of sale, manufacture, assembly, or use. Specific examples are provided for illustrating how to make and use the compositions, and examples of methods are not intended to be a representation that given embodiments have, or have not, been made or tested. Equivalent changes, modifications and variations of some embodiments, materials, compositions and methods can be made within the scope of the appended claims, with substantially similar results.

As used herein, the words “include,” “contain,” and their variants, are intended to be non-limiting, such that recitation of items in a list is not necessarily to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Moreover, descriptions of various alternatives using terms such as “or” do not require mutual exclusivity unless clearly required by the context, and the indefinite articles “a” or “an” do not limit the subject to a single instance unless clearly required by the context.

Although the open-ended term “comprising,” as a synonym of non-restrictive terms such as including, containing, or having, is used herein to describe and claim embodiments, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosure of values and ranges of values for specific parameters, such as temperatures, molecular weights, weight percentages, etc., are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, disclosure of two or more ranges of values for a parameter, whether such ranges are nested, overlapping or distinct, may subsume all possible combination of ranges for the value that might be claimed using endpoints of the disclosed ranges. For example, if parameter X is exemplified herein to have values in the range of 1-10, or 2-9, or 3-8, Parameter X may be envisioned as having other ranges of values including 1-2, 1-3, 1-8, 1-9, 2-3, 2-8, 2-10, 3-9, 3-10, 8-9, 8-10, and 9-10.

The term “about,” as used herein, is intended to refer to deviations in a numerical quantity that may result from various circumstances, for example, through measuring or handling procedures in the real world; through inadvertent error in such procedures; through differences in the manufacture, source, or purity of compositions or reagents; from computational or rounding procedures; and other deviations as will be apparent by those of skill in the art from the context of this disclosure. For example, unless otherwise defined by the specification per se or by the context of the specification, the term “about,” with reference to a value, may refer to any number that would round to that value, based on a significant digit analysis. In such a circumstance, a value of “about 30%”, assuming the “3” is the only significant digit, could encompass from 25% to just below 35%. However, the context of the specification would limit that interpretation based on significant digits, so that the “about” ranges do not overlap. For example, if the specification discloses ranges that include “about 25%, about 30%, about 35%,” etc., about 30% in that context could encompass from 27.5% to just below 32.5%. Alternatively, the term “about” may refer to deviations that are greater or lesser than a stated value or range by ±10% of the stated value(s), as appropriate from the context of the disclosure. In such a circumstance, a value of “about 30%” may encompass from 27% to 33%. Whether or not modified by the term “about,” quantitative values recited herein include equivalents to the recited values, for example, deviations from the numerical quantity, as would be recognized as equivalent by a person skilled in the art in view of this disclosure.

The appended claims set forth novel and inventive aspects of the subject matter disclosed and described above, but the claims may also encompass additional subject matter not specifically recited in detail. For example, certain features, elements, or aspects may be omitted from the disclosure and claims, if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention, as defined by the appended claims.

1. A wound dressing comprising:
an absorbent layer capable of absorbing a given volume of fluid and comprising a first sorption zone comprising a first density of fibers and a second sorption zone comprising a second density of fibers higher than the first density;
a hydrophilic foam layer coupled to the absorbent layer; and
a backing layer extending beyond the absorbent layer and the hydrophilic foam layer to define a backing layer margin,
wherein, upon exposure of the absorbent layer to a saturation threshold of wound exudate, the first sorption zone expands to a first thickness and the second sorption zone expands to a second thickness that is visually distinguishable from the first thickness.

2. The wound dressing of claim 1, wherein an interface between the first sorption zone and the second sorption zone creates a topological design based on a differential between the first thickness and the second thickness.

3. The wound dressing of claim 1, wherein:
the hydrophilic foam layer is substantially elastically deformable under wound treatment conditions,
the absorbent layer is not elastically deformable under wound treatment conditions, and
the coupled combination of the hydrophilic foam layer and the absorbent layer is substantially elastically deformable under wound treatment conditions.

4. The wound dressing of claim 1, wherein the absorbent layer comprises:
(i) from about 45% to about 90% of cellulose ether fibers; and
(ii) from about 10% to about 55% of reinforcing fibers.

5. The wound dressing of claim 4, wherein the cellulose ether fibers are composed of at least one of carboxymethyl cellulose, carboxylethyl cellulose, hydroxyethyl cellulose, hydroxypropyl cellulose, and hydroxypropylmethyl cellulose.

6. The wound dressing of claim 5, wherein the reinforcing fibers are composed of a polyurethane gel, an amide polymer, an olefin polymer, a modified acrylamide polymer, or a combination or copolymer thereof.

7. The wound dressing of claim 4, wherein the absorbent layer further comprises from about 10% to about 40% of an alginate, chitosan, chitin, a guar gum, pectin, a starch derivative, a cellulose derivative, a glycosaminoglycan, a galactomannan, a chondroitin salt, heparin, collagen, oxidized regenerated cellulose (ORC), a heparin salt, hyaluronic acid, a hyaluronic acid salt, or a combination thereof.

8. The wound dressing of claim 1, wherein the foam layer is coupled to the absorbent layer by lamination.

9. The wound dressing of claim 1, wherein the backing layer is non-adherent and comprises a polyurethane.

10. The wound dressing of claim 1, wherein the absorbent layer comprises superabsorbent polymer fibers, non-woven fibers, or a combination thereof.

11. The wound dressing of claim 1, further comprising a non-adherent layer disposed on the foam layer opposite the absorbent layer.

12. The wound dressing of claim 11, wherein the non-adherent layer is perforated and comprises at least one of an alkyl acrylate polymer, an alkacrylate polymer, and an alkyl alkacrylate polymer.

13. The wound dressing of claim 12, wherein the alkyl acrylate polymer comprises an ethylene-methyl acrylate copolymer.

14. The wound dressing of claim 1, comprising an adherent layer coupled to the backing layer on at least a margin thereof, and optionally extending over a margin of the foam layer thereby fastening the adherent layer to the margin of the foam layer.

15. The wound dressing of claim 14, wherein the adherent layer comprises a hydrocolloid.

16. The wound dressing of claim 1, wherein the wound dressing has a width and a length perpendicular to the width, and wherein the wound dressing exhibits a substantially elastic response when exposed to about 20% strain relative to the length for about 10 minutes.

17. The wound dressing of claim 1, wherein a difference between the first density and the second density is based on a difference in degrees of needling respectively applied to the first sorption zone and the second sorption zone.

18. A method for manufacturing a wound dressing which, upon exposure to a saturation threshold of fluid, differentially expands in a visually distinguishable manner, the method comprising:
providing a backing layer, a hydrophilic foam layer, and an absorbent layer capable of absorbing a given volume of wound exudate;
differentially treating the absorbent layer using a needling apparatus, so as to attain a first needled zone and a second needled zone, each needled zone having differential needling levels such that, upon exposure to a saturation threshold of wound exudate, the first needled zone expands to a first thickness and the second needled zone expands to a second thickness that is visually distinguishable from the first thickness; and
coupling the differentially treated absorbent layer to the foam layer, such that the backing layer extends beyond the absorbent layer to define a margin.

19. A method of reducing edema for a wound surrounded by tissue, the method comprising:
providing a wound dressing having:
an absorbent layer capable of absorbing a given volume of fluid and comprising a first sorption zone comprising a first density of fibers and a second sorption zone comprising a second density of fibers higher than the first density, so that upon exposure of the absorbent layer to a saturation threshold of wound exudate, the first sorption zone expands to a first thickness and the second sorption zone expands to a second thickness that is visually distinguishable from the first thickness;
a hydrophilic foam layer coupled to the absorbent layer;
a backing layer extending beyond the absorbent layer and the hydrophilic foam layer to define a backing layer margin;
an adherent layer coupled to the backing layer on at least a margin thereof, and optionally extending over a margin of the foam layer thereby fastening the adherent layer to the margin of the foam layer;
positioning the wound dressing over the wound, such that at least a portion of the adherent layer contacts the tissue.

20. (canceled)

21. The method of claim 19, wherein the wound is proximal to a knee joint or an elbow joint.

22. The method of claim 19, further comprising:
sealing the wound dressing within a sealed space adjacent to the wound; and
applying a negative pressure to the sealed space.

23. (canceled)
